(19) United States
(12) Patent Application Publication
(10) Pub. No.: US 2026/0069594 A1
(43) Pub. Date: Mar. 12, 2026

(54) USE OF AN AGENT CAPABLE OF INHIBITING THE ACTIVATION OF MAIT CELLS FOR THE TREATMENT OF RHEUMATOID ARTHRITIS

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); UNIVERSITÉ PARIS CITÉ, Paris (FR); ASSISTANCE PUBLIQUE-HÔPITAUX DE PARIS, Paris (FR)

(72) Inventors: Jérôme AVOUAC, Paris (FR); Agnès LEHUEN-MONTEIRO, Paris (FR); Manon LESTURGIE, Paris (FR); Virginie GONZALEZ, Paris (FR)

(21) Appl. No.: 19/107,796

(22) PCT Filed: Aug. 31, 2023
(86) PCT No.: PCT/EP2023/073962

§ 371 Date: Feb. 28, 2025

(30) Foreign Application Priority Data
Sep. 2, 2022 (EP) 22306303.3

(51) Int. Cl. A61K 31/519; A61K 31/11; A61K 31/60; A61P 19/02; C07K 16/28

(52) U.S. Cl. CPC A61K 31/519; A61K 31/11; A61K 31/60; A61P 19/02; C07K 16/2833

(57) ABSTRACT

Rheumatoid arthritis (RA) is the most common form of inflammatory rheumatism involving small joints which are the seat for swelling and pain with structural damage, responsible for functional disabilities if no treatment is proposed. The inventors show that circulating MAIT cells were reduced and exhibited an activated and anti-apoptotic phenotype in RA patients compared to healthy controls. MAIT cell levels were also found to be increased in the synovial fluid as compared with the peripheral blood, suggesting that circulating MAIT cell deficiency is due to the migration of MAIT cells into the joint. Severity of arthritis induced by mBSA was reduced in mice depleted in MAIT cells. This data suggest that MAIT cells contribute to exacerbation of arthritis. Thus the present invention relates to the use of an agent capable of inhibiting the activation of MAIT cells for the treatment of rheumatoid arthritis

### FIELD OF THE INVENTION

The present invention is in the field of medicine, in particular rheumatology.

### BACKGROUND OF THE INVENTION

Rheumatoid arthritis (RA) is the most common form of inflammatory rheumatism involving small joints which are the seat for swelling and pain with structural damage, responsible for functional disabilities if no treatment is proposed. To avoid joint destruction and disabilities, treatment should aim at reaching a target, such as remission or low disease activity in all patients, suppressing systemic and joint inflammation as soon as possible. According to the American, European and French recommendations for management of RA, methotrexate (MTX) should be started first, as soon as a diagnosis of RA is made (C. Gaujoux-Viala, L. Gossec, A. Cantagrel, M. Dougados, B. Fautrel, X. Mariette, H. Nataf, A. Saraux, S. Trope, B. Combe, Recommendations of the French Society for Rheumatology for managing rheumatoid arthritis. Joint Bone Spine 81, 287-297 (2014)). But, despite its potent anti-inflammatory action, with or without corticosteroids, MTX leads to low disease activity states in 25-50% of patients with early RA and to remission states in only 10-20% of RA patients at 6-12 months (E. B. Lee, R. Fleischmann, S. Hall, B. Wilkinson, J. D. Bradley, D. Gruben, T. Koncz, S. Krishnaswami, G. V. Wallenstein, C. Zang, S. H. Zwillich, R. F. van Vollenhoven, Tofacitinib versus methotrexate in rheumatoid arthritis. N Engl J Med 370, 2377-2386 (2014)). Advancement over the last few decades of our understanding of RA pathophysiology has led to the development of new treatments designed to act against a precise therapeutic target. These new molecules called ‘biologics’ include tumor necrosis factor-alpha (TNFα) blocking agents such as adalimumab, certolizumab, etanercept, golimumab and infliximab; an interleukin-1-receptor antagonist (IL1-Ra, anakinra); an inhibitor of the co-stimulation pathways implicated in T-lymphocyte activation [CTLA4-Ig or abatacept (ABA)]; a monoclonal antibody (mAb) binding to CD20 expressed on B cells (rituximab); a monoclonal antibody binding IL-6R (tocilizumab) (J. S. Smolen, D. Aletaha, Rheumatoid arthritis therapy reappraisal: strategies, opportunities and challenges. Nat Rev Rheumatol 11, 276-289 (2015)). All these agents have proven efficacy in reducing joint inflammation and, thus pain, and limiting or stopping joint destruction in association with the anchor drug MTX. However, no response to these treatments is obtained in approximately ˜30% of RA patients, and the response to all these medications is highly variable from one patient to another. This is probably because RA is a syndrome grouping several entities with different pathophysiological mechanisms, with a spectrum ranging from the most ‘inflammatory’ forms, susceptible of responding to immunotherapies directed against proinflammatory cytokines (anti-TNFα, anti-IL6, etc), to the most ‘autoimmune’ forms, able to respond better to anti-cellular therapies (rituximab and ABA). There is thus a need for identifying new targets for the treatment of RA.

Mucosal-associated invariant T (MAIT) cells are a subset of innate-like immune cells found in peripheral blood, intestinal mucosa, and abundantly in human liver. MAIT cells express an invariant T cell receptor α chain, the Vα7.2-Jα33 chain in humans. MAIT cells can produce IFN-γ, granzyme B (GrB), and IL-17; are restricted by the major histocompatibility complex class I-related molecule MR1; and are activated by cells infected by different microorganisms. Vitamin B2 (riboflavin) metabolites produced by bacteria and yeasts are required to generate MAIT cell-activating ligands. It has been recently shown that MAIT cells can also have various deleterious and protective functions in autoimmune, inflammatory and metabolic diseases. MAIT cell involvement in a large spectrum of pathological conditions makes them attractive targets for potential therapeutic approaches (Toubal A, Nel I, Lotersztajn S, Lehuen A. Mucosal-associated invariant T cells and disease. Nat Rev Immunol. 2019 October; 19(10):643-657.).

### SUMMARY OF THE INVENTION

The present invention is defined by the claims. In particular, the present invention relates to the use of an agent capable of inhibiting the activation of MAIT cells for the treatment of rheumatoid arthritis.

### DETAILED DESCRIPTION OF THE INVENTION

The first object of the present invention relates to a method of treating rheumatoid arthritis in a patient in need thereof comprising administering to the patient a therapeutically effective amount of an agent capable of inhibiting the activation of MAIT cells.

As used herein, the term “rheumatoid arthritis” or “RA” has its general meaning in the art and refers to a systemic autoimmune inflammatory pathology, characterized by causing persistent synovitis of the joints, causing their progressive destruction, generating different degrees of deformity and functional disability. The process starts with the intervention of humoral and cell factors, which generate inflammation mediating molecules, attract and activate peripheral blood cells, causing proliferation and activation of the synoviocytes, invading and destroying joint cartilage, subchondral bone, tendons and ligaments. Examples of physiological indexes of RA include symmetrical joint swelling and pain on passive movement, which are characteristics, but not unchangeable, of RA. In RA, the disease activity can be measured according to the standards recognized in the art. The “Disease Activity Score” or “DAS” is a measure of the activity of RA. In Europe the DAS is the recognized standard in research and clinical practice. The following parameters are included in the calculation (Van Gestel A M, Prevoo M L L, van't Hof M A, et al. Development and validation of the European League Against Rheumatism response criteria for RA. Arthritis Rheum 1996; 39:34-40): Number of joints tender to the touch (TEN), Number of swollen joints (SW), Erythrocyte sedimentation rate (ESR), Patient assessment of disease activity (VAS; mm).

As used herein, the term “patient” denotes a mammal. In some embodiments, a patient refers to any patient (preferably human) afflicted with RA. In some embodiments, the patient is resistant to methotrexate or is resistant to a biologic selected from the group consisting of tumor necrosis factor-alpha (TNFα) blocking agents such as adalimumab, certolizumab, etanercept, golimumab and infliximab; an interleukin-1-receptor antagonist (IL1-Ra, anakinra); inhibitors of the co-stimulation pathways implicated in T-lymphocyte activation [CTLA4-Ig or abatacept (ABA)]; monoclonal antibodies (mAb) binding to CD20 expressed on B cells (rituximab); and monoclonal antibodies binding IL-6R (tocilizumab).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “MAIT cells” or “Mucosal-Associated Invariant T cells” refers to a population of T cells present in mammals, preferably humans, that display an invariant TCR alpha chain comprising Vα7.2-Jα33 (in humans), a CDR3 of constant length, and a limited number of Vβ segments together with an activated phenotype (CD44) (see, e.g., Lantz and Bendelac. 1994. J. Exp Med. 180:1097-106; Tilloy et al., J. Exp. Med., 1999, 1907-1921; Treiner et al. (2003) Nature 422:164-169, the entire disclosures of each of which are herein incorporated by reference). MAIT cells are generally CD8⁺ (expressing mostly the homodimeric form of CD8αα) or CD4⁻/CD8⁻(DN), and are restricted by the non-classical MHC class I molecule MR1. For the purposes of the present invention, any T cells that express the invariant Vα7.2-Jα33 alpha TCR chain are considered to be MAIT cells. Typically, the alpha chain is associated with an invariant CDR3 and with either Vβ2 or Vβ13.

As used herein, the expression “agent capable of inhibiting the activation of MAIT cells” refers to any refers to any molecule that under cellular and/or physiological conditions is capable of inhibiting the pro-inflammatory functions of MAIT cells.

In some embodiments, the agent is a small organic molecule. Inhibitors of MAIT cells are known in the art and typically include those described in Corbett, A. J. et al. T-cell activation by transitory neo-antigens derived from distinct microbial pathways. Nature 509, 361-365 (2014); and Keller A N et al. Drugs and drug-like molecules can modulate the function of mucosal-associated invariant T cells Nat Immunol. 2017 April ; 18(4):402-411. Other examples include those described in the International Patent Application WO 2014005194. In some embodiments, the inhibitor is selected from the group consisting of 6-formyl pterin, acetyl-6-formylpterin (Ac-6-FP), 3-formylsalicylic acid (3-F-SA), 5-formylsalicylic acid (5-F-SA) and 2-hydroxy-1-naphthaldehyde (2-OH-1-NA).

In some embodiments, the agent is an antibody. As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference).

In some embodiments, the agent is an antibody that depletes MAIT cells (i.e. a “depleting antibody”). As used herein, the term “depletion” with respect to MAIT cells, refers to a measurable decrease in the number of MAIT cells in the subject. The reduction can be at least about 10%, e.g., at least about 20%, 30%, 40%, 50%, 60%, 70%, 80%, 90%, 95%, 96%, 97%, 98%, 99%, or more. In some embodiments, the depleting antibody binds to a cell surface marker of MAIT cells, preferentially a specific cell surface marker of MAIT cells. In some embodiments, the agent is an anti-Vα7.2-Jα33 depleting antibody such as described in the international patent publication WO2008087219. In some embodiments, the depleting antibody mediates antibody-dependent cell-mediated cytotoxicity. As used herein the term “antibody-dependent cell-mediated cytotoxicity” or ‘ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. In some embodiments, the depleting antibody is an IgG1 antibody. In some embodiments, the depleting antibody is an IgG3 antibody.

In some embodiments, the agent is an antibody that blocks the presentation of antigenic ligands (e.g. microbial vitamin B metabolites) by MR1. In some embodiments, the antibody blocks the interaction between MR1 the Vα7.2-Jα33 receptors. In some embodiments, the antibody binds to MR1. These antibodies are thus referred to as “neutralizing” or “inhibitory” or “blocking” antibodies. In some embodiments, the agent is an anti-MR1 neutralizing antibody. In some embodiments, the agent is an anti-Vα7.2-Jα33 neutralizing antibody such as described in the international patent publication WO2008087219. Such antibodies are useful, inter alia, for decreasing MAIT immune cell activity.

As use herein, the expression “therapeutically effective amount” refers to an amount effective of the agent, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above.

Typically, the agent of the present invention is administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1: Circulating MAIT cells deficiency in rheumatoid arthritis (RA).

PBMCs (5×10⁶) were collected from healthy donors (n=41) and from rheumatoid arthritis patients (n=43) and were analyzed by flow cytometry. Circulating MAIT cells were identified as CD161^{high} Vα7.2⁺ T cells. In the participants with rheumatoid arthritis (RA), MAIT cells among CD3⁺ cells was reduced, as compared to healthy donors (median (IQR) of MAIT cells proportion: 0.41% (0.19-0.97) in RA patients compared to 2.55% (1.5-2.6) in healthy donors, p<0.0001).

Each symbol represents a single individual and small horizontal lines represent median. Test: non-parametric two-tailed Mann-Whitney test. IQR: Interquartile range. MAIT cells: Mucosal Associated Invariant T Cells. PBMC: Peripheral Blood Mononuclear Cells. RA: Rheumatoid Arthritis.

FIG. 2: Correlation between circulating MAIT cells deficiency and disease activity.

We next investigated whether MAIT cell frequency was associated with clinical variables in RA patients (n=43). Disease activity was assessed with the Disease Activity Score 28 (DAS28) CRP which is calculated based on joint swelling and tenderness, systemic inflammation markers (CRP) and patient feedback. MAIT cells frequency correlated negatively with disease activity (r=−0.38, p=0.011). Thus, the more active the patient, the more pronounced the deficiency of circulating MAIT cells.

Each symbol represents a single individual. Test: Spearman's correlation test. CRP: C reactive protein. DAS28: Disease Activity Score. IQR: Interquartile range. MAIT cells: Mucosal Associated Invariant T cells. RA: Rheumatoid Arthritis.

FIG. 3: Accumulation of MAIT cells in synovial fluid compared to blood in rheumatoid arthritis patients.

PBMCs (5×10⁶) and synovial fluid mononuclear cells (5×10⁶) were collected from rheumatoid arthritis patients (n=15) and were analyzed by flow cytometry. Circulating and synovial fluid MAIT cells were identified as CD161^{high} Vα7.2⁺ cells. The proportion of synovial fluid MAIT cells among CD3⁺ cells was higher, as compared to circulating MAIT cells (median (IQR) of MAIT cells proportion: 0.65% (0.2-4.7) in synovial fluid compared to 0.23% (0.09-0.88) in blood, p=0.025).

Each symbol represents a single individual. Test: two-tailed Wilcoxon matched-pairs test. IQR: Interquartile range. MAIT cells: Mucosal Associated Invariant T cells. PBMC: Peripheral Blood Mononuclear Cells. SF: Synovial Fluid.

FIG. 4: Activated profile and loss of CD56 in synovial fluid MAIT cells compared to peripheral blood MAIT cells in rheumatoid arthritis patients.

We next investigated the phenotype of circulating MAIT cells in healthy donors and rheumatoid arthritis, and phenotype of synovial fluid MAIT cells in rheumatoid arthritis patients. In rheumatoid arthritis patients, circulating MAIT cells displayed an activated phenotype indicated by markedly increased CD69⁺ MAIT cell frequency, as compared to healthy donors (median (IQR) of CD69⁺ MAIT cell frequency: 22.2 % (11.6-30.4) compared to 9.8% (4-14.7) in healthy donors, p=0.0005, FIG. 4A) and increased CD25⁺ MAIT cell frequency, as compared to healthy donors (median (IQR) of CD25⁺ MAIT cell frequency: 2.2% (0.3-18.5) vs. 0.57% (0.29-0.78), p=0.03, FIG. 4B). Moreover, in RA patients, synovial fluid MAIT cells were even more activated, as compared to the blood, indicated by 75% increase in the CD69⁺ MAIT cell frequency in synovial fluid compared to the blood (median (IQR) of CD69⁺ MAIT cell frequency: 86.1% (74.5-94.1) in synovial fluid compared to 22.2% (11.6-30.4) in blood, p=0.0001, FIG. 4A). This was consistent with a strong trend towards increasing of CD25⁺ MAIT cells, another T cell activation marker, in synovial fluid, as compared to the blood (median (IQR) of CD25⁺ MAIT cell frequency: 7.4% (1.2-34.5) compared to 2.2% (0.3-18.5), p-0.057, FIG. 4B). Finally, we observed a loss of the adhesion molecule CD56 in synovial fluid, as compared to the blood (median (IQR) of CD56⁺ MAIT cell frequency: 38.9% (26.5-55.4) compared to 62% (49.3-74), p=0.0012, FIG. 4C) which could suggest a migration of MAIT cells from the blood to the inflamed tissue, the joint.

Each symbol represents a single individual and small horizontal lines represent median. Test: non-parametric two-tailed Mann-Whitney test between healthy donor blood and RA blood; two-tailed Wilcoxon matched-pairs test between RA blood and RA synovial fluid. IQR: Inter quartile range. MAIT cells: Mucosal Associated Invariant T Cells. RA: Rheumatoid Arthritis.

FIG. 5: Reduction in the severity of arthritis in mice depleted in MAIT cells.

Arthritis was induced by intra-articular injection of methylated bovine serum albumin (mBSA) in MR1^{-/-} (depleted in MAIT cells) and wild type C57BL/6 mice, previously immunized with mBSA. We included 9 mice in each group, only females, which were 6 to 8 weeks old. Mice were daily evaluated for signs of joint inflammation, assessed by measuring tarsal thickness and scored on a scale of 0-4, where 0=no change, 1=local swelling and redness of digits, 2=mild swelling and redness, 3=severe swelling and redness, 4=necrosis. Mice were sacrificed on day 8, and the paws were separated, fixed with 4% paraformaldehyde and decalcified for histopathological assessment. All mice developed arthritis, with a peak of inflammation at day 1. Wild type mice had a higher clinical score than MR1 KO mice (A), with a significant increase in area under curve (B) (mean AUC in MR1 KO group at 4.7 compared to 9.1 in wild type group, p=0.0064).

Values are the mean+/−SEM of 9 mice in each group. Test: Student T test. AUC: Area Under Curve. MAIT cells: Mucosal Associated Invariant T cells. mBSA: methylated bovin serum albumin. KO: Knock-out. MR1: MHC class I related-1 molecule.

FIG. 6: Persistence of arthritis more pronounced in mice with MAIT cell overexpression.

Arthritis was induced by intra-articular injection of methylated bovine serum albumin (mBSA) in Vα19 transgenic mice (with 10 times more MAIT cells than wild type mice) and wild type C57BL/6 mice, previously immunized with mBSA. We included 7 mice in Vα19 transgenic group and 11 wild type, only females, which were 6 to 14 weeks old. Mice were daily evaluated as described previously. All mice developed arthritis, with a peak of inflammation at day 1. Although there were no differences in the clinical score between the two groups in the first part of the experiment (mean AUC at 4.6 in Vα19 transgenic mice group compared to 4.3 in wild type group, p=0.7), we observed a persistence of arthritis more marked after day 5 in the Vα19 transgenic mice (mean AUC in Vα19 transgenic mice at 1.1 compared to 0.55, p=0.025).

Values are the mean+/−SEM of mice in each group. Test: Student T test. AUC: Area Under Curve. MAIT cells: Mucosal Associated Invariant T cells. mBSA: methylated bovin serum albumin.

FIG. 7: Strong trend to a reduction in the severity of arthritis in mice treated with a molecule inhibiting MAIT cell activation.

Arthritis was induced by intra-articular injection of methylated bovine serum albumin (mBSA) in wild type mice, treated with Acetyl-6-formylpterin (Ac6FP), a non-activating ligand of MAIT cells or PBS, previously immunized with mBSA. Ac6FP or PBS were injected twice a week in intraperitoneal. We included 6 wild type mice in each group, only males, which were 8 weeks old. Mice were daily evaluated as described previously. All mice developed arthritis, with a peak of inflammation at day 1. Mice treated by Ac6FP had a lower clinical score than mice treated by PBS (A), with a decrease in area under curve (B) (median AUC in group treated by Ac6FP at 3, vs. 5.5 in PBS group, p=0.07).

Values are the mean+/−SEM of 6 mice in each group. Test: Student T test. Ac6FP: Acetyl-6-formylpterin. AUC: Area Under Curve. MAIT cells: Mucosal Associated Invariant T cells. mBSA: methylated bovin serum albumin. PBS: Dulbecco's Phosphate Buffered Saline.

### EXAMPLE

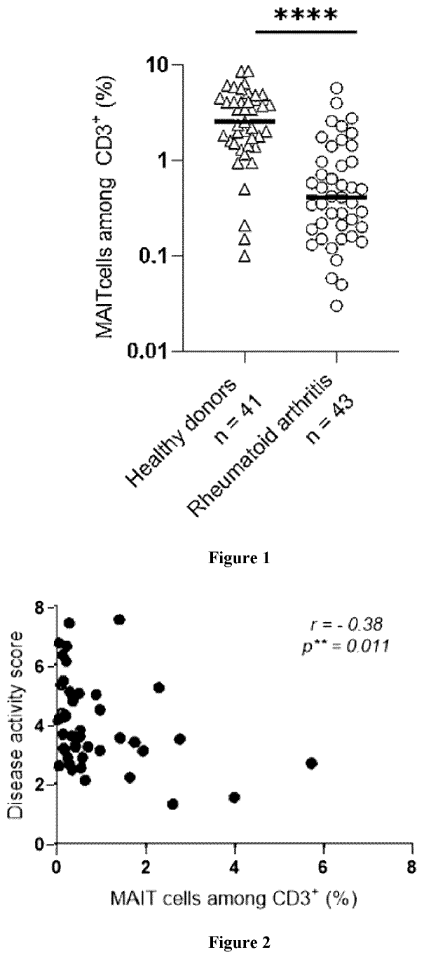

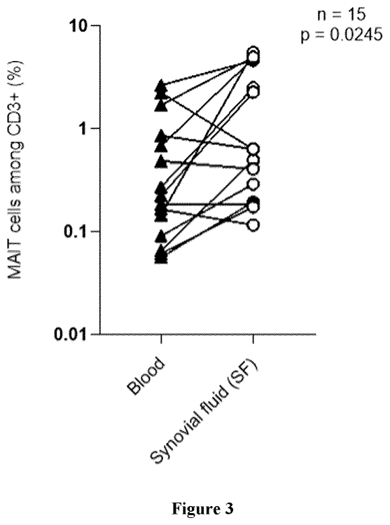

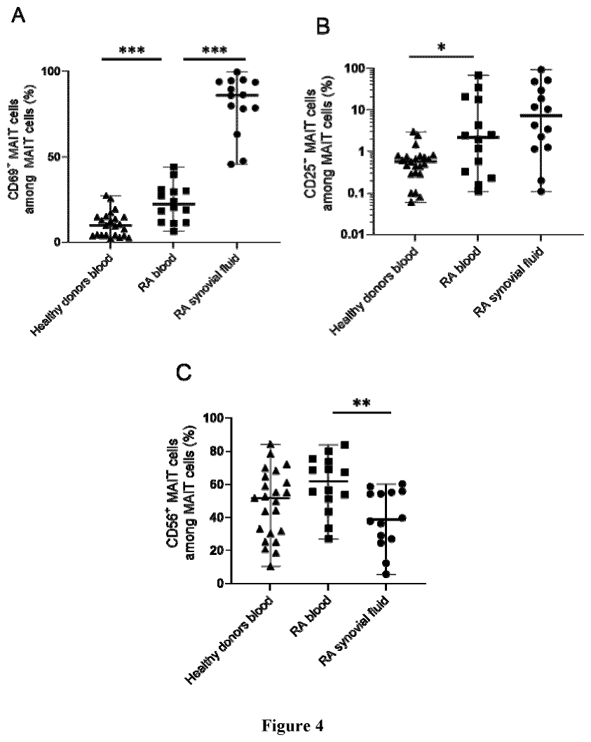

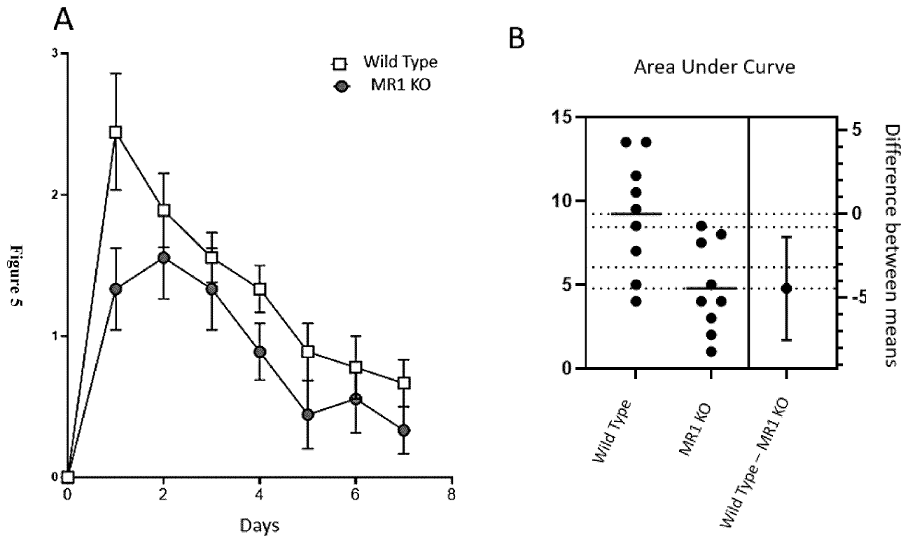

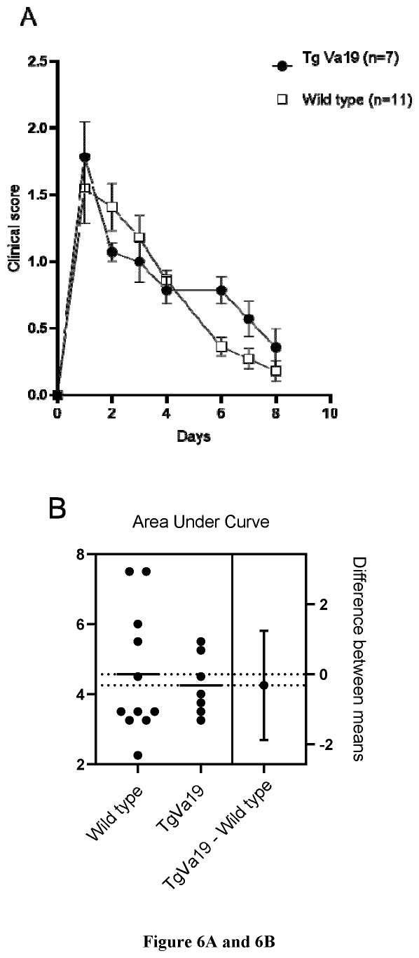

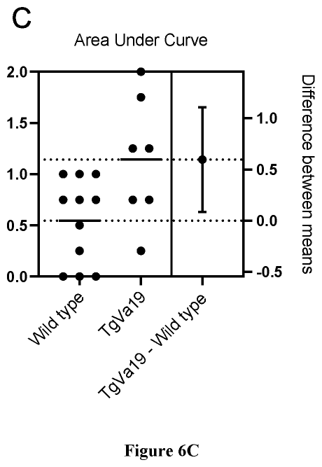

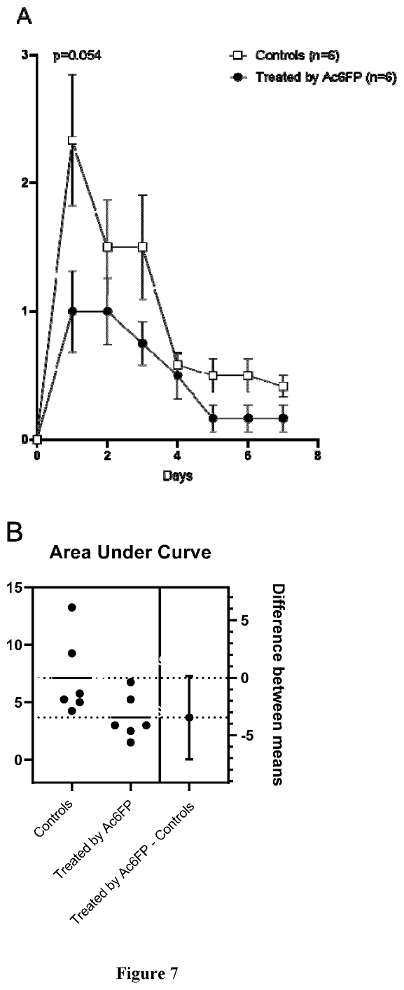

Circulating MAIT cells were reduced (FIGS. 1 and 2) and exhibited an activated phenotype (FIGS. 4A and 4B) in RA patients compared to healthy controls. MAIT cell levels were also found to be increased in the synovial fluid as compared with the peripheral blood (FIG. 3), with an activated profile (FIG. 4A) and loss of adhesion molecule, CD56 (FIG. 4C), suggesting that circulating MAIT cell deficiency is due to the migration of MAIT cells into the joint. Severity of arthritis induced by mBSA was reduced in mice depleted in MAIT cells (FIG. 5) while persistence of arthritis was more pronounced in mice with MAIT cell overexpression (FIG. 6). Moreover, the severity of arthritis trend to be reduced in mice treated with a molecule inhibiting MAIT cell activation (FIG. 7). All of these data suggest that MAIT cells contribute to exacerbation of arthritis.

### REFERENCES

Throughout this application, various references describe the state of the art to which this invention pertains. The disclosures of these references are hereby incorporated by reference into the present disclosure.

1. A method of treating rheumatoid arthritis in a patient in need thereof comprising administering to the patient a therapeutically effective amount of an agent capable of inhibiting the activation of MAIT cells.

2. The method of claim 1 wherein the agent is a small organic molecule.

3. The method of claim 2 wherein the agent is selected from the group consisting of 6-formyl pterin, acetyl-6-formylpterin (Ac-6-FP), 3-formylsalicylic acid (3-F-SA), 5-formylsalicylic acid (5-F-SA) and 2-hydroxy-1-naphthaldehyde (2-OH-1-NA).

4. The method of claim 1 wherein the agent is an antibody.

5. The method of claim 4 wherein the antibody is an antibody that depletes MAIT cells.

6. The method of claim 4 wherein the antibody is an antibody that blocks the presentation of antigenic ligands by MR1.

7. The method of claim 6 wherein the antibody blocks the interaction between MR1 the V 2-J 33 receptors.

8. The method of claim 7 wherein the antibody binds to MR1.
